(19) United States
(12) Patent Application Publication
(10) Pub. No.: US 2020/0306459 A1
(43) Pub. Date: Oct. 1, 2020

(54) USE OF FILLER IN AMPUTATED EXTREMITIES TO SIMULATE SOFT TISSUE COVERAGE

(71) Applicant: Andreas Nikolis, Westmount (CA)

(72) Inventor: Andreas Nikolis, Westmount (CA)

(21) Appl. No.: 16/363,259

(22) Filed: Mar. 25, 2019

(51) Int. Cl. A61M 5/32; A61L 27/20; A61L 27/52

(52) U.S. Cl. CPC A61M 5/32; A61L 27/20; A61L 2430/34; A61L 2400/06; A61L 27/52

(57) ABSTRACT

Hyaluronic acid is used for simulating subcutaneous soft tissue in a residual limb of a subject.

### Field of the Invention

The present invention generally relates to a method and use of a dermal filler, such as hyaluronic acid, to provide simulated additional soft tissue coverage at a residual limb after an amputation.

### BACKGROUND OF THE INVENTION

Amputation of a digit or limb can be a debilitating condition resulting in prolonged time-off work and chronic sequelae. The Comission de la santé et de la sécurité du travail (CSST), the worker's compensation agency of Quebec, reported that over 8400 cases of injuries to the digits were accepted in 2014.[1] This accounted for 10% of all cases that were accepted by the program. Injuries to the digits are second only to cases involving the back/spine. A cost-analysis on work injuries in Quebec, demonstrated that on average, patients were on work-leave for an average of 49 days which amounted to a total mean cost of $26,000 per injury.[2]

Although the data is unavailable for Canada, in the United States, traumatic finger amputations total approximately 45,000 cases a year, of which over 90% are treated without a replantation of the amputated digit.[3, 4] A systematic review demonstrated that on average 43% of patients reported cold intolerance, 37% had pain at the site of the trauma, 32% had paresthesias/hypoesthesia and 27% had hypersensitivity.[5] Neuroma's had the lowest prevalence at 1%.

Patients also find difficulty with their hand function given the lack of soft-tissue coverage over the bony amputated stump. The sensitivity of the bony prominence hinders grasping of objects, which ultimately serves as an obstacle for a return to work or resuming normal activities. A study on the determinants of returning to work in patients with hand injuries and disorders demonstrated that pain is a major independent negative predictor.[6] Unfortunately, hand surgeons are limited in their capacity to treat these complications, as additional surgical interventions are limited and unlikely to improve outcomes.

There is a strong incentive from both a societal and healthcare perspective to find solutions for chronic pain and sensitivity in amputated digits or members.

Soft tissue fillers, such as hyaluronic acid, have the potential to provide volume through minimally invasive techniques. They have served to treat wrinkles, volume loss and scars, to name of a few of their clinical applications.[7-10]

Hyaluronic acid is a naturally-occurring component of the extracellular matrix. It is a glycosaminoglycan polymer consisting of repeat disaccharide units of glucuronic acid and N-acetylglucosamine. Hyaluronic acid polymers vary considerably in length, with the weight influencing the characteristics and potential uses of the polymer.

Typically, hyaluronic acid dermal fillers consist of long chain higher molecular weight hyaluronic acid. Often the hyaluronic acid is cross linked with a chemical, such as 1,4-butanedioldiglycidyl ether (BDDE) or divinyl sulfone (DVX). For use as a dermal filler, suspension of the hyaluronic acid in a solution produces a homogeneous gel or a suspension of particles in a gel carrier.

The commercial demand for such dermal fillers has resulted in the use of non-animal based hyaluronic acid, which has been produced by a bacterial fermentation process. Variability in methods used to manufacture hyaluronic acid-based dermal fillers have given rise to differences in properties such as degree of cross-linkage, particle size and concentration.

There have been few reports of using a filler in an amputated digit or limb. A group of researchers have used the augmentation properties of HA on amputated lower limbs of diabetic patients.[11] They compared the tissue properties of heels between patients treated with HA and compared to native amputated heels. They reported HA increased tissue thickness and enhanced tissue resilience. Of course, this was an incomparable treatment for a different pathology, but it did demonstrate some potentially effective uses of fillers for the treatment of amputation symptoms. The digits are unique as the extra volume in this region will allow for a better “cushion” between bone and overlying skin, as well as the ability to possibly mask neurologic symptoms from exposed nerve endings. Another group used hyaluronic acid during surgical procedures in an attempt to alleviate symptoms associated with a neuroma in upper and lower extremities. [12]

### REFERENCES

1. Comission de la santé et de la sécurité du travail du Québec. Statistiques anuelles 2014. 2015.
2. Institut de recherche Robert-Sauvé en santé et en sécurité du travail. Les coats des lésions professionnelles au Québec, 2005-2007. 2014 (Rapport R-769).
3. Centers for Disease Control and Prevention. Work-related injury statistics query system. 2014.
4. Friedrich, J. B., et al., Epidemiology of upper extremity replantation surgery in the United States. J Hand Surg Am, 2011. 36(11): p. 1835-40.
5. Yuan, F., et al., A Systematic Review of Outcomes after Revision Amputation for Treatment of Traumatic Finger Amputation. Plast Reconstr Surg, 2015. 136(1): p. 99-113.
6. Opsteegh, L., et al., Determinants of return to work in patients with hand disorders and hand injuries. J Occup Rehabil, 2009. 19(3): p. 245-55.
7. Duranti, F., et al., Injectable hyaluronic acid gel for soft tissue augmentation. A clinical and histological study. Dermatol Surg, 1998. 24(12): p. 1317-25.
8. Elliott, L., R. M. Rashid, and M. Colome, Hyaluronic acid filler for steroid atrophy. J Cosmet Dermatol, 2010. 9(3): p. 253-5.
9. Khan, F., K. Richards, and R. M. Rashid, Hyaluronic acid filler for a depressed scar. Dermatol Online J, 2012. 18(5): p. 15.
10. Richards, K. N. and R. M. Rashid, Twenty-four-month persistence of hyaluronic acid filler for an atrophic scar. J Cosmet Dermatol, 2011. 10(4): p. 311-2.
11. Hsu, C. C., et al., Determination of the augmentation effects of hyaluronic acid on different heel structures in amputated lower limbs of diabetic patients using ultrasound elastography. Ultrasound Med Biol, 2012. 38(6): p. 943-52.
12. Monacelli, G., et al. [Surgical treatment of painful amputation neuromas with hyaluronic acid gel. Preliminary study of six patients]. G Chir. 2007 Jan.-Feb.; 28(1-2):25-8.

### SUMMARY OF THE INVENTION

According to an aspect of the present invention, there is provided a use of hyaluronic acid for simulating subcutaneous soft tissue in a residual limb of a subject.

According to another aspect of the present invention, there is provided a method for simulating subcutaneous soft tissue in a residual limb after amputation. The method includes introducing hyaluronic acid into the residual limb of the subject.

According to a further aspect of the present invention, there is provided a kit for providing simulated subcutaneous soft tissue coverage at a residual limb after an amputation. The kit includes hyaluronic acid, at least one syringe, and at least one needle.

### BRIEF DESCRIPTION OF THE DRAWINGS

The invention will now be described in more detail having regard to the drawings in which:

FIG. 1 illustrates injection of hyaluronic acid according to an embodiment of the present invention;

FIG. 2A illustrates a residual limb before injection with hyaluronic acid according to an embodiment of the present invention; and

FIG. 2B illustrates the residual limb shown in FIG. 2A after injection with hyaluronic acid according to an embodiment of the present invention.

### DETAILED DESCRIPTION OF THE INVENTION

The disclosure relates to uses of hyaluronic acid, and methods, to provide simulated soft tissue coverage to a subject at a residual limb after an amputation. A kit comprising components for providing simulated soft tissue coverage to a subject at a residual limb after an amputation is also disclosed.

The subject can be any mammal, preferably a human of any age, gender or race. The subject will typically have a residual limb or stump as a result of an accident or an amputation. Preferably, the subject is experiencing some discomfort in the residual limb, such as thermal or mechanical sensitivity.

In one embodiment, the hyaluronic acid can be cross-linked or not cross-linked. Cross-linked hyaluronic acid is preferred, as it is typically more durable, has greater longevity, and may be more water absorbent. Although, any pharmaceutically or cosmetically acceptable hyaluronic acid can be used in the disclosed methods, uses, and kits. For example, the hyaluronic acid compositions and formulations sold as Revanesse Ultra®, JUVEDERM®, JUVEDERM® VOLUMA®, JUVEDERM® Ultra XC, JUVEDERM® VOLIFT®, JUVEDERM® VOLBELLA®, BELOTERO® HYDRO, BELOTERO® SOFT, BELOTERO® BALANCE, BELOTERO® INTENSE, BELOTERO® VOLUME, RESTYLANE®, RESTYLANE® LYFT, RESTYLANE® REFYNE, RESTYLANE® DEFYNE, RESTYLANE® SILK, RESTYLANE® VITAL, RESTYLANE® VOLUME, and RESTYLANE® KYSSE, is a non-exhaustive exemplary list that of hyaluronic acid-based products that can be used according to the present invention.

The formulation of the hyaluronic acid is preferably in the form of an injectable gel, and preferably has a high viscosity. The hyaluronic acid is preferably monophasic, but may also be biphasic. It may be derived from animal sources, but can be a non-animal based hyaluronic acid, such as that commonly made by bacterial fermentation from e.g. streptococci bacteria.

Although there is not one particular hyaluronic acid that is ideal for all patient subtypes, the most commonly used fillers used for this indication would be that of a moderate HA concentration, i.e. 18-24 mg/ml.

Hyaluronic acid presents an interesting opportunity to provide subcutaneous padding for amputated stumps. Without being bound by any explanation, it is thought that the hyaluronic acid provides additional physical padding that can reduce both bony contact at the stump and direct pressure on a neuroma. The hyaluronic acid may also serve to recruit additional macromolecules to the site. Providing some artificial padding to the residual limb will create additional protection to surrounding nerves, which may minimize or eliminate the abnormal pain and sensitivity. Introduction of the hyaluronic acid filler also serves the added benefit of increasing the aesthetic outcomes. After introduction, the residual limb will appear “plumped up”, which adds a more natural, fatty, appearance. Also, given that areas of residual limbs with poor soft tissue padding are typically more difficult to fit comfortably with a prosthesis, the disclosed use and method could be useful to patients who require the use of a prosthetic. Overall this treatment option may improve: appearance, patient satisfaction, hypersensitivity and cold intolerance, pain management, and active use. Patients can then regain their ability to pursue their hobbies, return to work or continue their rehabilitation to desensitize any persistent painful scar, which was not amenable to therapy previously due to a sensitive bony surface.

The present invention may be applied to extremities lacking appropriate soft tissue coverage, including residual limbs produced through accident or amputation. In particular, the present application may be applied to e.g. digital or toe tip amputations, failed re-implant procedures, amputations following diabetic complications, amputations following tumour excisions. It should be noted that all extremities are eligible as potential treatment areas.

According to one embodiment, a hyaluronic acid is introduced, preferably through injection, into a residual limb of a patient to provide simulated soft tissue coverage. Preferably, the hyaluronic gel is a high viscosity, cross-linked hyaluronic acid gel. Injection of the hyaluronic acid occurs via syringe and needle. The hyaluronic acid may come preloaded in a syringe, or the syringe will need to be loaded with the hyaluronic acid, such as from a vial. In one embodiment, the syringe will be attached to a 27 to 30-gauge needle, however, the size of the needle can be selected based on the volume of hyaluronic acid that is to be injected, and the viscosity of the formulation. A formulation with a high viscosity may need a smaller gauge needle.

Preferably, the hyaluronic acid is in the form of a high viscosity gel. The concentration of the hyaluronic acid that is to be injected may vary, but is preferably injected at a concentration of about 20-28 mg/ml, and more preferably about 25 mg/ml. A higher viscosity of the injectable formulation is preferred, as this will provide a more durable padding, and should minimize subcutaneous movement of the gel.

The volume of hyaluronic acid that will be injected into site of interest will vary based upon the size of the residual limb and/or the size of the bony structure at the limb, and the underlying structure of the tissue under treatment. It will be appreciated that an injection at a wrist after a hand amputation will require a larger volume of hyaluronic acid than an injection at a finger after a digit amputation. For example, a smaller site, such as after a partial amputation of a finger, may equate to approximately a 5 mm by 5 mm area, could be sufficiently injected with a volume of about 0.05 cc to 0.5 cc of hyaluronic acid.

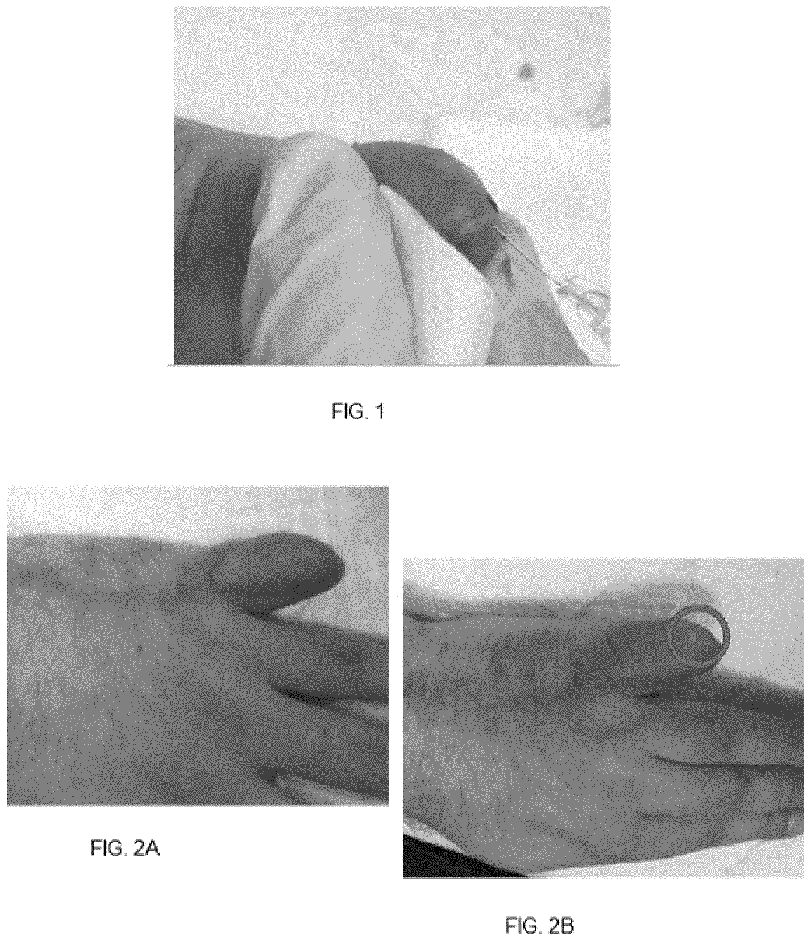

In one embodiment, in order to maximize effectiveness, the site of injection of the hyaluronic acid should be at an area with maximum depression and the least amount of pre-existing soft tissue volume (See FIG. 1). The area of injection will take into account the adherence of skin to underlying bony structures. A single injection per area is recommended. Although if necessary, the needle can be angled to allow for dispersion of the hyaluronic acid in the surrounding region, or subsequent injections can be done.

Preferably, the depth of the injection should reach around or into the periosteum of the underlying bony structure. This may require a mild injection force. Alternatively, the hyaluronic acid may be injected into the subcutaneous tissue surrounding the bony structure of the residual limb. The injection will likely cause the skin to expand to allow for the volume placement of the hyaluronic acid, with may become more pronounced if subsequent injections are required (See FIGS. 2A and 2B).

Evaluation of the hyaluronic acid injection can be done to assess if subsequent injections are required, which may take many forms. For example, prior to the first injection, an assessment may be performed to establish a baseline index, such as by having the subject identify a certain level on a pain index, observing a range of motion of the residual limb, and/or observing if the subject is able to complete a certain task with the residual limb. Following the first injection, the subject could be re-evaluated between e.g. 2 to 4 weeks post-injection. At this follow up visit, the same assessment should be performed and compared to the results of the baseline assessment. If desired improvement has not been obtained, an appropriate skin cushion has not been generated, and/or the residual limb does not have the desired cosmetic appearance, a second injection can be undertaken.

In one embodiment, there is provided a kit. The kit includes hyaluronic acid for injection to provide simulated additional soft tissue coverage at a residual limb after an amputation. The kit may comprise the hyaluronic acid, either pre-loaded in a syringe and/or in a vial, and the kit may contain multiple pre-loaded syringes and/or vials. The hyaluronic acid is preferably provided at 25 mg/ml of a high viscosity hyaluronic acid gel formulation, such as Revanesse Ultra. The kit may contain brief instructions suggesting injection into the mid- to deep-dermis of the residual limb.

For example, the kit may contain a biocompatible, biodegradable, non-pyrogenic, sterile, injectable viscoelastic clear colorless hydrogel based on bioresorbable cross-linked hyaluronic acid at 22-28 mg/mL concentration. The gel is delivered in a pre-filled disposable glass syringe. Each syringe is fitted with a luer lock adaptor, a plunger rod, a rubber stopper tip cap, and a finger grip. Each kit contains two 1.0 mL syringes of the hyaluronic acid along with two 0.5-inch 27 to 30 gauge sterile needles in a molded rigid PVC tray. The syringe may be labeled with the product name, the manufacturer, lot number, and expiration date. There may be a removable portion of the label, which can be affixed to the patient record.

### EXAMPLE

A 40-year-old male working as a manual labourer was involved in a crushing accident that resulted in amputation of the left pinky finger. After an attempt to re-plant the digit failed, treatment with revision amputation and local flap was pursued to improve soft tissue coverage. The patient reported being in constant pain, and was left unable to work due to his injury.

One year following the accident and approximately six months after the last surgery was performed, the following injection technique was performed to aid with symptoms:

Under local anesthetic, the patient was injected with 0.2 cc of product in the region of the finger lacking soft tissue protection (See FIG. 1). Results demonstrating improvement were found immediately (See FIGS. 2A and 2B). After anesthetic wore off, the patient reported less pain and significantly improved ability to palpitate the region without any discomfort. Prior to injection, the patient was unable to perform a movement with the digit in the absence of pain and discomfort. When the patient returned after one week, they were able to perform the movement with no pain or discomfort. The patient only required one session to achieve results, which lasted approximately six months.

1. Use of hyaluronic acid for simulating subcutaneous soft tissue in a residual limb of a subject.

2. The use according to claim 1, wherein the hyaluronic acid is cross-linked.

3. The use according to claim 1, wherein the hyaluronic acid is in the form of an injectable gel.

4. The use according to claim 1, wherein the hyaluronic acid is non-animal based.

5. The use according to claim 1, wherein the hyaluronic acid is at a concentration of 22 to 28 mg/ml.

6. The use according to claim 1, wherein the hyaluronic acid is formulated for subcutaneous injection at the residual limb.

7. The use according to claim 1, wherein the residual limb is a finger.

8. The use according to claim 1, wherein the subject is human.

9. A method for simulating subcutaneous soft tissue in a residual limb of a subject, comprising:
introducing hyaluronic acid into the residual limb of the subject.

10. The method according to claim 9, wherein the hyaluronic acid is introduced into the residual limb via injection with a needle.

11. The method according to claim 10, further comprising a subsequent injection of hyaluronic acid into the residual limb of the patient.

12. The method according to claim 11, wherein the injection reaches the periosteum of the underlying bony structure.

13. The method according to claim 12, wherein the hyaluronic acid is cross linked.

14. The method according to claim 12, wherein the hyaluronic acid is in the form of an injectable gel.

15. The method according to claim 14, wherein the hyaluronic acid is non-animal based.

16. The method according to claim 15, wherein the hyaluronic acid is at a concentration of 22 to 28 mg/ml.

17. The method according to claim 16, wherein the hyaluronic acid is for subcutaneous injection at the residual limb.

18. The method according to claim 17, wherein the residual limb is a finger.

19. The method according to claim 18, wherein the subject is human.

20. A kit for simulating subcutaneous soft tissue in a residual limb of a subject, the kit comprising:
hyaluronic acid;
at least one syringe; and
at least one needle.

21. The kit according to claim 20, wherein the hyaluronic acid is cross-linked.

22. The kit according to claim 21, wherein the hyaluronic acid is in the form of an injectable gel.

23. The kit according to claim 22, wherein the hyaluronic acid is non-animal based.

24. The kit according to claim 23, wherein the hyaluronic acid is at a concentration of 22 to 28 mg/ml.

25. The kit according to claim 24, wherein the at least one needle is 30 gauge or smaller.

26. The kit according to claim 25, wherein the hyaluronic acid is pre-loaded in the at least one syringe.

27. The kit according to claim 26, further comprising instructions directing injection of the hyaluronic acid into the mid to deep dermis, or to the periosteum of the underlying bony structure, of the residual limb.
